(19) United States
(12) Patent Application Publication
(10) Pub. No.: US 2016/0304520 A1
(43) Pub. Date: Oct. 20, 2016

(54) An improved process for preparing Linagliptin and its key Intermediates

(71) Applicant: WOCKHARDT LIMITED, Aurangabad O, (IN)

(72) Inventors: Naveen REDDY, Hyderabad (IN); Damodara K. NAIDU, Hyderabad (IN); Vivek Thakaram RAUT, Ahmednagar (IN); Bhatraju Srinivasa RAO, Godavri (IN); Keshav DEO, Vadodara (IN)

(21) Appl. No.: 14/902,257

(22) PCT Filed: Jul. 8, 2014
(86) PCT No.: PCT/IB2014/062943

§ 371 Date: Dec. 30, 2015

(30) Foreign Application Priority Data
Jul. 11, 2013 (IN) 2337/MUM/2013
Jul. 4, 2014 (IN) 2181/MUM/2014

(51) Int. Cl. C07D 473/04

(52) U.S. Cl. CPC C07D 473/04

(57) ABSTRACT

The present invention relates to a process for the preparation of Linagliptin or a pharmaceutically acceptable slat thereof. Further aspects of the present invention relates to process for the preparation of Linagliptin key intermediate, having purity more than 98.0%.

### FIELD OF INVENTION

The present invention relates to a process for the preparation of Linagliptin or a pharmaceutically acceptable salt thereof. Further it relates to process for the preparation of Linagliptin key intermediates, 8-bromo xanthine and Boc-Linagliptin having purity more than 98.0%.

### BACKGROUND OF THE INVENTION

Linagliptin is chemically known 8-[(3R)-3-aminopiperidin-1-yl]-7-(but-2-yn-1-yl)-3-methyl-1-[(4-methylquinazolin-2-yl)methyl]-3,7-dihydro-1H-purine-2,6-dione and is structurally represented by formula (I):

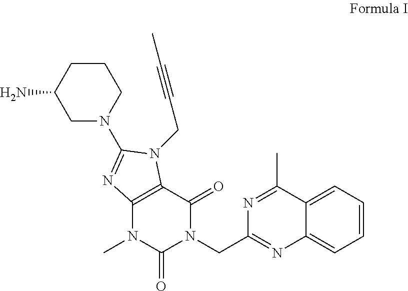 [SMILES: CC#CCn1c(N2CCC[C@@H](N)C2)nc2c1c(=O)n(Cc1nc(C)c3ccccc3n1)c(=O)n2C]

Linagliptin is approved for the treatment of Diabetes and is available in the market as Tradjenta® with the strength 5 mg of Tablet for oral administration.

U.S. Pat. No. 7,407,955 describes Linagliptin and process for the preparation thereof. Further, Matthias et al., in Journal of Medicinal Chemistry 2007 50(26) Pages 6450-6453, discloses a process for the preparation of Linagliptin from Boc protected Linagliptin.

The process disclosed in the US '955 and Matthias article for Boc protected Linagliptin involves condensation of 8-chloro xanthine compound with —(R)-Boc aminopiperidine in presence potassium carbonate.

The reported process suffers one or the other problems like yield and purity due to the selection of suitable solvent and reaction condition. Hence, there is a need for a simple process for making large scale quantities of Linagliptin or a pharmaceutically acceptable salt thereof.

The inventors of the present invention surprisingly found that the positive improvement, for example, yield and purity, due to the use of organic base than the inorganic base for the condensation reaction to provide Boc protected Linagliptin.

The inventors of the present invention also noticed that Linagliptin in yield and purity could be prepared by using substantially pure intermediates in suitable solvent such as amide and water or mixture thereof. The intermediates 8-bromo-7-(but-2-ynyl)-3-methyl-1-((4-methylquinazolin-2-yl)methyl)-1H-purine-2,6(3H,7H)-dione (referred to herein as “8-bromo xanthine”) of Formula-II and (R)-1-(7-(but-2-ynyl)-2,3,6,7-tetrahydro-3-methyl-1-((4-methylquinazolin-2-yl) methyl)-2,6-dioxo-1H-purin-8-yl)piperidin-3-ylcarbamate (referred to herein as “Boc-Linagliptin”) of Formula-III can be used to prepare highly pure Linagliptin in the subsequent reaction steps.

### SUMMARY OF THE INVENTION

The present invention provides an improved process for the preparation of Linagliptin or pharmaceutically acceptable salt thereof. Further, the present invention provides the process for the preparation of substantially pure Linagliptin key intermediates, e.g. bromopurine and Boc-Linagliptin, having purity more than 98.0%.

The present invention provides a process for the preparation of Linagliptin of Formula I or its pharmaceutically acceptable salt, which includes steps of condensation of 2-(chloromethyl)-4-methylquinazoline compound of Formula A or its pharmaceutically acceptable salt with 8-bromo-7-(but-2-ynyl)-3-methyl-1H-purine-2,6(3H,7H)-dione of Formula B or its pharmaceutically acceptable salt, in presence of a suitable base in suitable solvent to obtain 8-bromo xanthine of compound of Formula II or its pharmaceutically acceptable salt. Purification of 8-bromo xanthine of Formula II as obtained in step (a) in the suitable solvent. Condensation of 8-bromo xanthine compound of Formula II or its pharmaceutically acceptable salt with 3-(R)-Boc aminopiperidine of Formula C or its pharmaceutically acceptable salt, using a suitable base in presence of a suitable solvent to obtain Boc-Linagliptin of Formula III or its pharmaceutically acceptable salt. Deprotection of Boc-Linagliptin using triflouroacetic acid in presence of dichloromethane to obtain crude Linagliptin. Acid and base treatment to crude Linagliptin yields substantially pure Linagliptin.

The present invention provides a process for preparing substantially pure 8-bromo xanthine Formula II or pharmaceutically acceptable salt thereof:

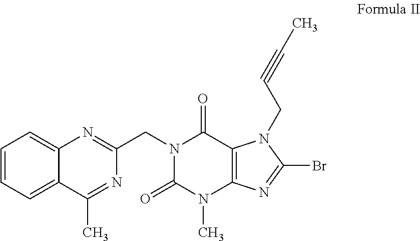 [SMILES: CC#CCn1c(Br)nc2c1c(=O)n(Cc1nc(C)c3ccccc3n1)c(=O)n2C]

and its conversion to Linagliptin or pharmaceutically acceptable salt thereof.

The present invention also provides a process for preparing substantially pure Boc-Linagliptin of Formula III or pharmaceutically acceptable salt thereof:

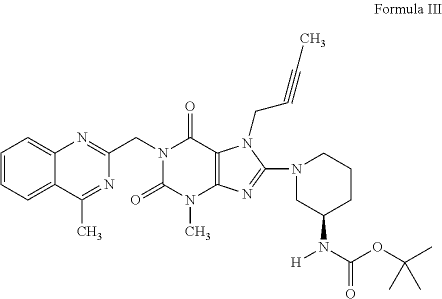 [SMILES: CC#CCn1c(N2CCC[C@@H](NC(=O)OC(C)(C)C)C2)nc2c1c(=O)n(Cc1nc(C)c3ccccc3n1)c(=O)n2C]

and its conversion to Linagliptin or pharmaceutically acceptable salt thereof.

In another aspect, the present invention specifically provides the process for the preparation of intermediate of Linagliptin of Formula III or its pharmaceutically acceptable salt, which includes condensation of 8-bromo xanthine compound of Formula II or its pharmaceutically acceptable salt with 3-(R)-Boc aminopiperidine of Formula C or its pharmaceutically acceptable salt using an organic base in the presence of a solvent.

In another aspect, the present invention provides Linagliptin or a pharmaceutically acceptable salt thereof having purity greater than or equal to 99%.

In another aspect, the present invention provides highly pure Linagliptin or a pharmaceutically acceptable salt thereof substantially free of impurities, e.g. N-(1-(7-(but-2-ynyl)-2,3,6,7-tetrahydro-3-methyl-1-((4-methylquinazolin-2-yl)methyl)-2,6-dioxo-1H-purin-8-yl)piperidin-3-yl)-2,2,2-trifluoroacetamide (referred to herein as “TFA-impurity”)

In another aspect, there is provided a composition comprises Linagliptin or a pharmaceutically acceptable salt thereof of the present invention and at least one pharmaceutically acceptable carrier or excipient.

### DESCRIPTION OF THE INVENTION

For purposes of the present invention, the following terms are defined below.

The term “substantially pure” as used herein, unless otherwise defined, the compound that has purity of greater than about 97% or greater than about 98%.

The intermediates and starting materials of the present invention may be used as free bases or its salts.

In an aspect, the present invention provides a process for the preparation of Linagliptin of Formula I

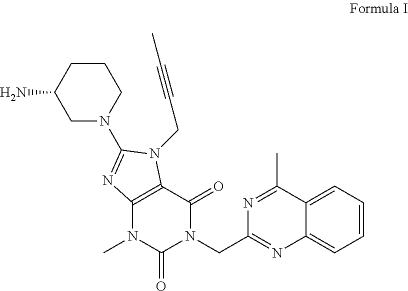 [SMILES: CC#CCn1c(N2CCC[C@@H](N)C2)nc2c1c(=O)n(Cc1nc(C)c3ccccc3n1)c(=O)n2C]

or its pharmaceutically acceptable salt, which includes steps of
a) condensation of 2-(chloromethyl)-4-methylquinazoline compound of Formula A

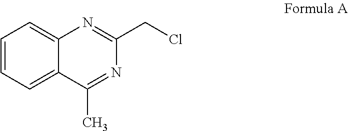

or its pharmaceutically acceptable salt with 8-bromo-7-(but-2-ynyl)-3-methyl-1H-purine-2,6(3H,7H)-dione of Formula B

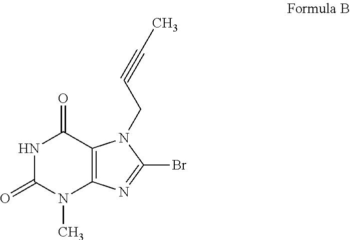 [SMILES: CC#CCn1c(Br)nc2c1c(=O)[nH]c(=O)n2C]

or its pharmaceutically acceptable salt, in presence of base in a suitable solvent to obtain 8-bromo xanthine of compound of Formula II or its pharmaceutically acceptable salt

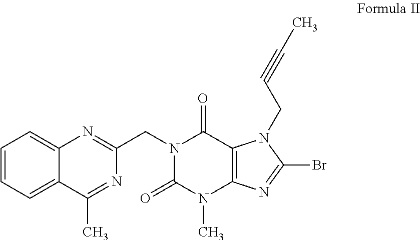 [SMILES: CC#CCn1c(Br)nc2c1c(=O)n(Cc1nc(C)c3ccccc3n1)c(=O)n2C]

b) purification of 8-bromo xanthine of Formula II as obtained in step (a) in the suitable solvent.
c) condensation of 8-bromo xanthine compound of Formula II or its pharmaceutically acceptable salt with 3-(R)-Boc aminopiperidine of Formula C

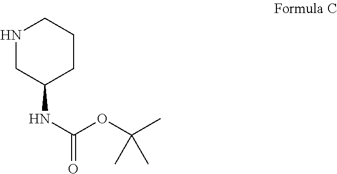

or its pharmaceutically acceptable salt, in presence of a suitable base in a suitable solvent to obtain Boc-Linagliptin of Formula III

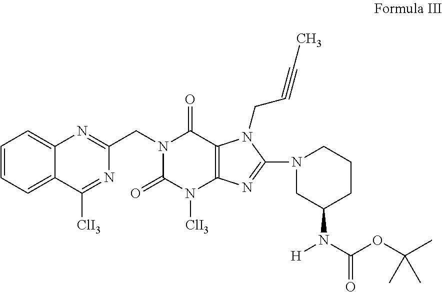

or its pharmaceutically acceptable salt
d) deprotection of Boc-Linagliptin of step c) using triflouroacetic acid in presence of dichloromethane to obtain crude Linagliptin.
e) treatment with suitable acid base to crude Linagliptin obtained in step (d) to obtain substantially pure Linagliptin.

In an another aspect, the present invention provides a process for the preparation of substantially pure 8-bromo xanthine of Formula II or its pharmaceutically acceptable salt,

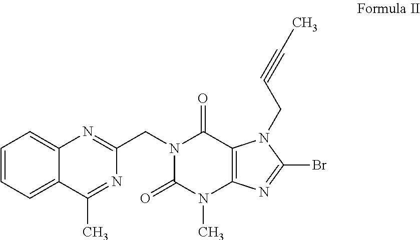 [SMILES: CC#CCn1c(Br)nc2c1c(=O)n(Cc1nc(C)c3ccccc3n1)c(=O)n2C]

which includes step of
a) condensation of 2-(chloromethyl)-4-methylquinazoline compound of Formula A

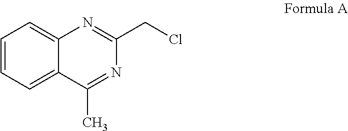

or its pharmaceutically acceptable salt with 8-bromo-7-(but-2-ynyl)-3-methyl-1H-purine-2,6(3H,7H)-dione of Formula B

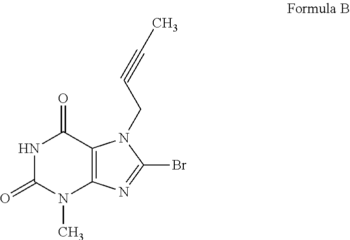 [SMILES: CC#CCn1c(Br)nc2c1c(=O)[nH]c(=O)n2C]

or its pharmaceutically acceptable salt, in presence of base in a suitable solvent.
b) purification of 8-bromo xanthine of Formula II obtained in step (a) in the suitable solvent.

In an another aspect, the present invention provides a process for the preparation of substantially pure Boc-Linagliptin of Formula III

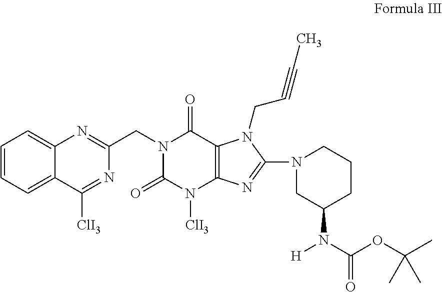

or its pharmaceutically acceptable salt, which includes condensation of 8-bromo xanthine compound of Formula II or its pharmaceutically acceptable salt with 3-(R)-Boc aminopiperidine of Formula C

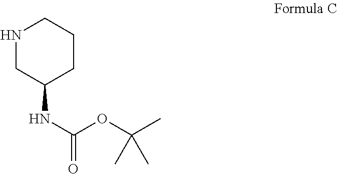

or its pharmaceutically acceptable salt in presence of a suitable base in a suitable solvent.

In another aspect, the present invention provides a process for the preparation of Linagliptin of Formula I

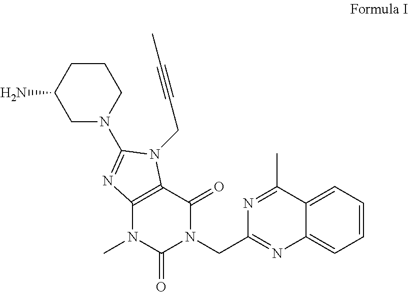 [SMILES: CC#CCn1c(N2CCC[C@@H](N)C2)nc2c1c(=O)n(Cc1nc(C)c3ccccc3n1)c(=O)n2C]

or its pharmaceutically acceptable salt, which includes steps of
a) condensation of 2-(chloromethyl)-4-methylquinazoline compound of Formula A

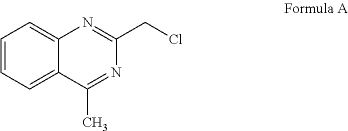

or its pharmaceutically acceptable salt with 8-bromo-7-(but-2-ynyl)-3-methyl-1H-purine-2,6(3H,7H)-dione of Formula B

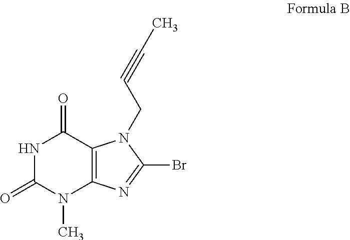 [SMILES: CC#CCn1c(Br)nc2c1c(=O)[nH]c(=O)n2C]

or its pharmaceutically acceptable salt, in presence of potassium carbonate base in dimethylacetamide solvent to obtain 8-bromo xanthine of compound of Formula II or its pharmaceutically acceptable salt.

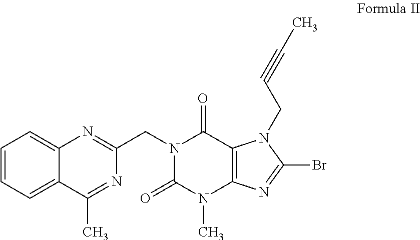 [SMILES: CC#CCn1c(Br)nc2c1c(=O)n(Cc1nc(C)c3ccccc3n1)c(=O)n2C]

b) purification of 8-bromo xanthine of Formula II as obtained in step (a) in dimethylformamide.
c) condensation of 8-bromo xanthine compound of Formula II or its pharmaceutically acceptable salt with 3-(R)-Boc aminopiperidine of Formula C

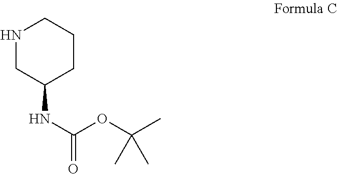

or its pharmaceutically acceptable salt, in presence of potassium carbonate base in dimethylacetamide solvent to obtain Boc-Linagliptin of Formula III

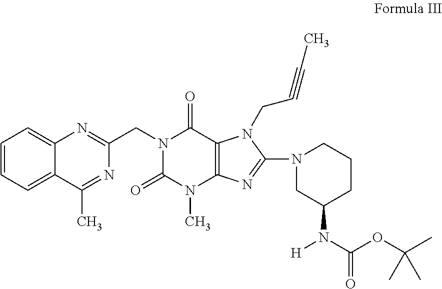 [SMILES: CC#CCn1c(N2CCC[C@@H](NC(=O)OC(C)(C)C)C2)nc2c1c(=O)n(Cc1nc(C)c3ccccc3n1)c(=O)n2C]

or its pharmaceutically acceptable salt
d) deprotection of Boc-Linagliptin of step c) using triflouroacetic acid in presence of dichloromethane to obtain crude Linagliptin.
e) treatment with suitable acid base to crude Linagliptin obtained in step (d) to obtain substantially pure Linagliptin.

In another aspect, the present invention provides a process for the preparation of Linagliptin or pharmaceutically acceptable salt thereof, which includes:
a) condensation of 8-bromo xanthine compound of Formula II or its pharmaceutically acceptable salt with 3-(R)-Boc aminopiperidine of Formula C or its pharmaceutically acceptable salt using an organic base such as N,N-Diisopropylethylamine in the presence of dimethyl acetamide to provide Boc protected Linagliptin; and
b) deprotection of Boc-Linagliptin of step a) using triflouroacetic acid in presence of dichloromethane to provide Linagliptin.

In another aspect, the present invention provides Linagliptin or a pharmaceutically acceptable salt thereof having purity greater than or equal to 99%.

In another aspect, the present invention provides highly pure Linagliptin or a pharmaceutically acceptable salt thereof substantially free of impurities, such as N-(1-(7-(but-2-ynyl)-2,3,6,7-tetrahydro-3-methyl-1-((4-methylquinazolin-2-yl)methyl)-2,6-dioxo-1H-purin-8-yl)piperidin-3-yl)-2,2,2-trifluoroacetamide (referred to herein as “impurity-A”)

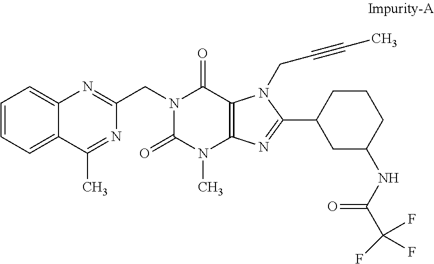 [SMILES: CC#CCn1c(C2CCCC(NC(=O)C(F)(F)F)C2)nc2c1c(=O)n(Cc1nc(C)c3ccccc3n1)c(=O)n2C]

The suitable base may be selected from inorganic base or organic base. The inorganic base includes but are not limited to sodium hydroxide, sodium carbonate, sodium bicarbonate, potassium carbonate, potassium hydroxide, potassium bicarbonate and the like; the organic base may be selected from triethyl amine, diisopropyl amine, methyl amine, pyridine, N-methyl pyrrolidine and the like.

The suitable solvent selected from the group comprising amides, water and their combination. The amides solvents are, such as dimethyl acetamide and dimethyl formamide and the like.

The condensation reaction may be conducted at elevated temperature of about 45° C. to about 110° C. temperature. The reaction may be maintained till to the completion of starting materials at elevated temperature, for about 2 hours or more.

After completion of the reaction, the reaction mixture may be quenched with water or acid and solid isolation by using suitable techniques, such as addition of organic solvent, antisolvent, concentrate, pH adjustment and cooling of reaction mixture or mass.

Organic solvent selected from group methanol, ethanol, isopropyl alcohol, n-butanol and the like. Antisolvent is selected from ethyl acetate, isopropyl acetate and the like. The term antisolvent is defined as the solvent for crystallization in which the solubility of the product to be crystallized is less and it is used for the complete crystallization of the product.

In another aspect, the present invention provides Linagliptin or a pharmaceutically acceptable salt thereof having purity greater than or equal to 99%.

The salt or pharmaceutically acceptable salt compounds of the present invention may include but are not limited to hydrochloride, hydrobromide, methane sulfonate, p-toluene sulfonate, trifluoroacetate, tartrate salt and the like.

The process of the present invention is depicted in the following Scheme A:

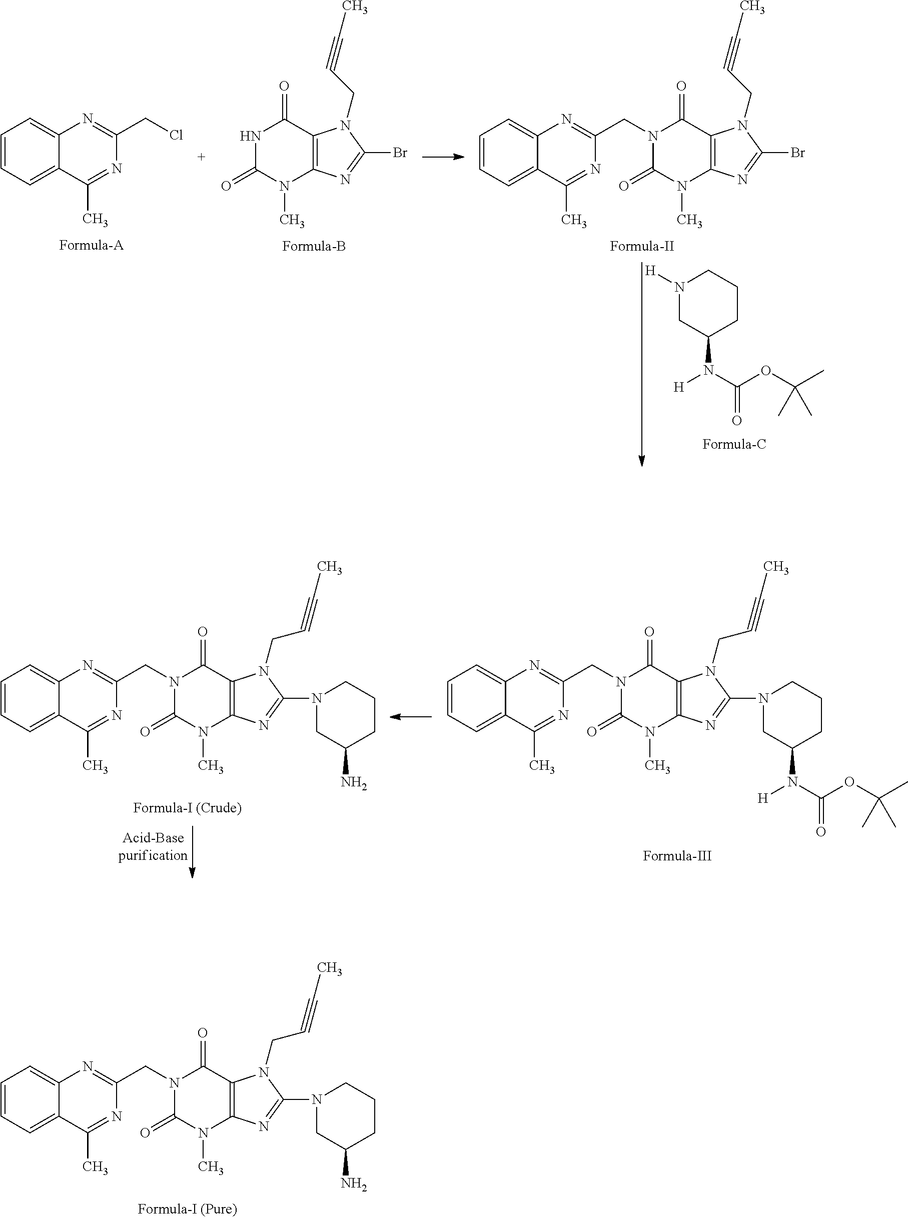

The resultant 8-bromo xanthine or its salt obtained from the present invention has purity greater than about 99% determined by HPLC method. The resultant Boc-Linagliptin or its salt obtained from the present invention has purity greater than about 98% determined by HPLC method. The yield of the 8-bromo xanthine or its salt may be greater than about 90%. The yield of the Boc-Linagliptin or its salt may be greater than about 90%.

In another aspect, the present invention provides the process for the preparation of intermediate of Linagliptin of Formula III:

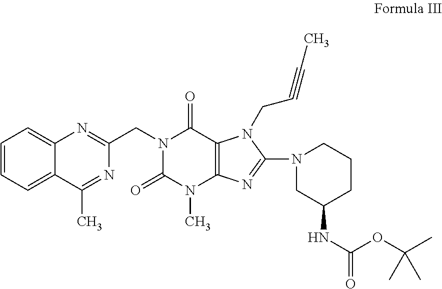 [SMILES: CC#CCn1c(N2CCC[C@@H](NC(=O)OC(C)(C)C)C2)nc2c1c(=O)n(Cc1nc(C)c3ccccc3n1)c(=O)n2C]

or its pharmaceutically acceptable salt, which includes condensation of 8-bromo xanthine compound of Formula II

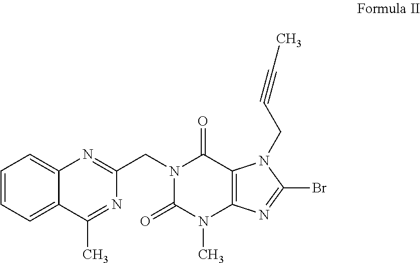 [SMILES: CC#CCn1c(Br)nc2c1c(=O)n(Cc1nc(C)c3ccccc3n1)c(=O)n2C]

or its pharmaceutically acceptable salt with 3-(R)-Boc aminopiperidine of Formula C

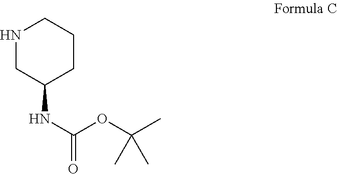

or its pharmaceutically acceptable salt using in presence of organic base in a suitable solvent.

The suitable organic base used for the condensation of 8-bromo xanthine compound of Formula II or its pharmaceutically acceptable salt with 3-(R)-Boc aminopiperidine of Formula C or its pharmaceutically acceptable salt is selected from Triethyl amine (Et₃N), trimethyl amine (Me₃N), pyridine, tributylamine, diisopropyl ethyl amine (DIPEA) in presence of a suitable solvent.

The suitable solvent for the condensation reaction of the present invention includes but are not limited to dimethyl acetamide, dimethyl formamide, dimethyl sulfoxide and the like. Other solvents may include alcohol such as methanol, ethanol, isopropanol and n-butanol; hydrocarbon such as n-hexane, n-heptane, cyclohexane and toluene; and their combination with dimethyl acetamide.

The starting material, 3-(R)-Boc aminopiperidine or its salt may be used 1 to 1.5 molar equivalents for the equivalent of 8-bromo xanthine compound of Formula II or its salt for preparing Boc-Linagliptin.

The base used for the condensation reaction may be 1 to 4 molar equivalents per the equivalent of the compound of Formula II or its salt.

The condensation reaction may be conducted at elevated temperature of about 45° C. to about reflux temperature. The reaction may be maintained till completion of the reaction, for example, to about 2 hours or more.

After completion of the reaction, the reaction mixture may be quenched with quenching agent, for example, water or acid, and then subjected for solid isolation by using suitable techniques, for example, slurry for a certain period of time, cooling, recrystallization and the like.

In another aspect, the present invention provides a process for the preparation of intermediate of Linagliptin of Formula III or its pharmaceutically acceptable salt, which includes condensation of 8-bromo xanthine compound of Formula II or its pharmaceutically acceptable salt with 3-(R)-Boc aminopiperidine of Formula C or its pharmaceutically acceptable salt using an organic base such as N,N-Diisopropylethylamine.

The process of the present invention is depicted in the following Scheme B:

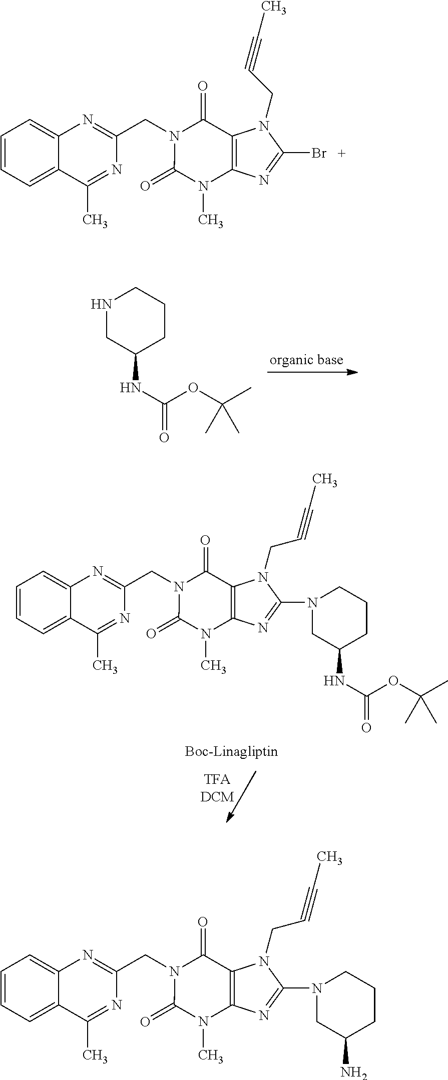 [SMILES: CC#CCn1c(Br)nc2c1c(=O)n(Cc1nc(C)c3ccccc3n1)c(=O)n2C ; CC(C)(C)OC(=O)N[C@@H]1CCCNC1 ; CC#CCn1c(N2CCC[C@@H](NC(=O)OC(C)(C)C)C2)nc2c1c(=O)n(Cc1nc(C)c3ccccc3n1)c(=O)n2C ; CC#CCn1c(N2CCC[C@@H](N)C2)nc2c1c(=O)n(Cc1nc(C)c3ccccc3n1)c(=O)n2C]

The salt or pharmaceutically acceptable salt compounds of the present invention may include but are not limited to hydrochloride, hydrobromide, methane sulfonate, p-toluene sulfonate, trifluoroacetate, tartrate salt and the like.

The obtained Boc-Linagliptin or its salt is used for the conversion to Linagliptin by the treatment of acid, for example, triflouroacetic acid, in presence of chlorinated solvent, for example, dichloromethane.

The resultant Boc-Linagliptin or its salt obtained from the present invention has purity greater than about 95% determined by HPLC method. The yield of the Boc-Linagliptin or its salt may be greater than about 92%

The present invention is further illustrated by the following example, which does not limit the scope of the invention. Certain modifications and equivalents will be apparent to those skilled in the art and are intended to be included within the scope of the present application.

### EXAMPLES

### Example-1

### Preparation of Bromopurine

To the stirring mixture of purine (200 g) in dimethylacetamide (1400 ml) were added quinazoline (147 g), potassium carbonate powder (140 g) at 25-30° C. The reaction mixture was heated to 80-85° C. for 10 hr. To the reaction mixture, water was charged (5600 ml). After addition of water, reaction mixture was cooled to 25-30° C. and maintained for 30 min. Filtered reaction mixture to give bromopurine (282 g) which is further purified from dimethyl formamide to give pure bromopurine (253 g).

HPLC Purity: 99.47%

### Example-2

### Preparation of Boc-Linagliptin

### Method A:

To the stirring mixture of Boc-Amlno piperidine (14 g) in dimethylacetamide (175 ml) were added potassium carbonate powder (31 g) and bromopurine (18 g) at 25-30° C. The reaction mixture was heated to 80-85° C. for 12 hr. To the reaction mixture, water was added (525 ml). After addition of water, reaction mass cooled to 35−40° C. and maintained for 30 min. Filtered reaction mixture to get Boc-Linagliptin (35 g).

HPLC Purity: 98.03%

### Method B:

Bromo xanthine (5 gm) and Boc-Amino piperidine (3.4 gm) were added into dimethyl acetamide (50 mL) at room temperature. Diisopropylethyl amine (5.6 gm) was added to the reaction mixture and then heated to 100-110° C. and maintain for 12 hours. After completion of reaction, reaction mixture was quenched with water (50 mL) and stirred at RT for 30 minutes. The precipitated material was filtered, washed with water (20 mL) and dried under vacuum at 65-70° C. to get Boc-Linagliptin (5.75 gm, 92% yield) with HPLC purity>96.76 (0.19% of bromopurine (starting material) observed).

### Example-3

### Preparation of Linagliptin-Crude

To the stirring solution of Boc-Linagliptin (35 gm) in dichloromethane (350 ml) at 15° C. was added trifluoroacetic acid (140 g) slowly. After addition of trifluoro acetic acid, reaction mixture was heated to 25-30° C. and maintained for 3 hour. Reaction mixture was cooled to 5° C. and water was added (200 ml) and adjusted the pH of the reaction mixture 9-10 using ammonia solution (110 g). Dichloromethane layer Separated and concentrated. The reaction mass is treated with Isopropyl Acetate (490 ml) and its partial removal by distillation and cooling to 10-15° C. yields crude Linagliptin (23 g).

HPLC Purity: 98.43%

### Example-4

### Preparation of Linagliptin

To the stirring mixture of crude Linagliptin (20 g) water (200 ml) was added concentrated hydrochloric acid (5 ml) at temperate 5−15° C. The reaction mixture was washed with dichloromethane and subsequently by ethyl acetate. The aqueous layer was treated with dichloromethane and the pH is adjusted to 10-11 using potassium carbonate solution at temperate below 15° C. Separated dichloromethane layer was washed with 25% sodium chloride solution. Further dichloromethane was distilled out and reaction mass was treated with Methanol (80 ml) partial removal of methanol and addition isopropyl acetate yields the Linagliptin which is filtered and dried. Yield 13 g.

HPLC Purity: 99.59%

1-24. (canceled)

25. A process for the preparation of Linagliptin of Formula I 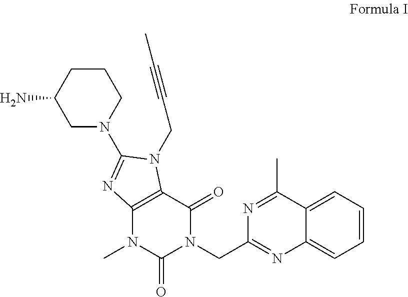 [SMILES: CC#CCn1c(N2CCC[C@@H](N)C2)nc2c1c(=O)n(Cc1nc(C)c3ccccc3n1)c(=O)n2C]
or its pharmaceutically acceptable salt, which comprises steps of
a) condensation of 2-(chloromethyl)-4-methylquinazoline compound of Formula A 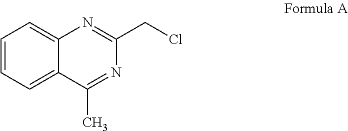
or its pharmaceutically acceptable salt with 8-bromo-7-(but-2-ynyl)-3-methyl-1H-purine-2,6(3H,7H)-dione of Formula B 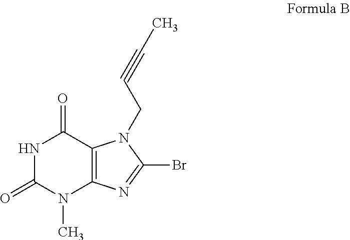 [SMILES: CC#CCn1c(Br)nc2c1c(=O)[nH]c(=O)n2C]
or its pharmaceutically acceptable salt, in presence of suitable base in suitable solvent and water to obtain 8-bromo xanthine of compound of Formula II or its pharmaceutically acceptable salt. 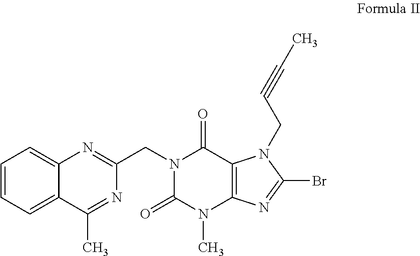 [SMILES: CC#CCn1c(Br)nc2c1c(=O)n(Cc1nc(C)c3ccccc3n1)c(=O)n2C]
b) purification of 8-bromo xanthine of Formula II obtained in step a) in suitable solvent to obtain substantially pure 8-bromo xanthine of Formula II
c) condensation of 8-bromo xanthine compound of Formula II or its pharmaceutically acceptable salt obtained in step b) with 3-(R)-Boc aminopiperidine of Formula C 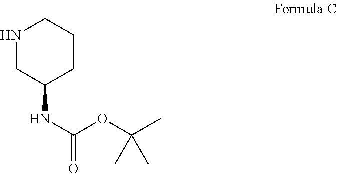
or its pharmaceutically acceptable salt, in presence of suitable base in suitable solvent to obtain substantially pure Boc-Linagliptin of Formula III 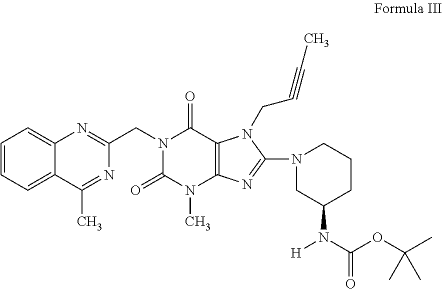 [SMILES: CC#CCn1c(N2CCC[C@@H](NC(=O)OC(C)(C)C)C2)nc2c1c(=O)n(Cc1nc(C)c3ccccc3n1)c(=O)n2C]
or its pharmaceutically acceptable salt
d) deprotection of Boc-Linagliptin of step c) using triflouroacetic acid in presence of dichloromethane to obtain crude Linagliptin.
e) optionally, treating step (d) product with suitable acid base to obtain substantially pure Linagliptin.

26. The process according to claim 25, wherein the suitable solvent is selected from the group comprising one or more dimethyl acetamide, dimethyl formamide and dimethyl sulfoxide or mixture thereof.

27. The process according to claim 26, wherein the suitable solvent used in step (b) and (c) is combination of dimethyl acetamide, dimethyl formamide, dimethyl sulfoxide and water.

28. The process according to claim 25, wherein suitable base used in step a) and c) comprising one or more of inorganic bases or organic bases or mixture thereof.

29. The process according to claim 25, wherein the substantially pure 8-bromo xanthine has the purity more than 99% by HPLC.

30. The process according to claim 25, wherein the substantially pure Boc-Linagliptin has the purity more than 98% by HPLC.

31. A substantially pure 8-bromo xanthine of Formula II 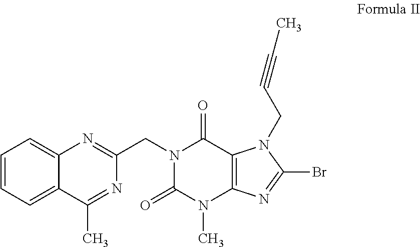 [SMILES: CC#CCn1c(Br)nc2c1c(=O)n(Cc1nc(C)c3ccccc3n1)c(=O)n2C]
or its pharmaceutically acceptable salt

32. A process for the preparation of Linagliptin of Formula I 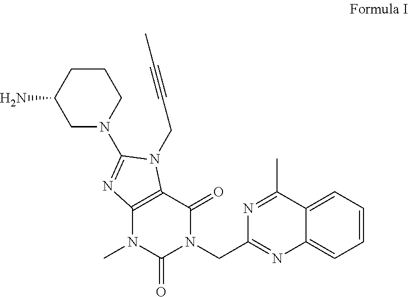 [SMILES: CC#CCn1c(N2CCC[C@@H](N)C2)nc2c1c(=O)n(Cc1nc(C)c3ccccc3n1)c(=O)n2C]
or its pharmaceutically acceptable salt, which comprising
a) condensing the substantially 8-bromo xanthine compound of Formula II 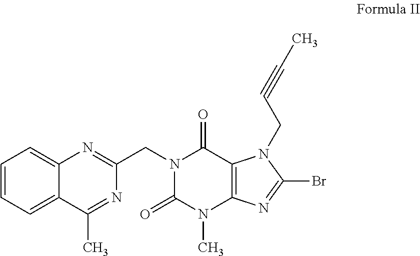 [SMILES: CC#CCn1c(Br)nc2c1c(=O)n(Cc1nc(C)c3ccccc3n1)c(=O)n2C]
or its pharmaceutically acceptable salt with 3-(R)-Boc aminopiperidine of Formula C 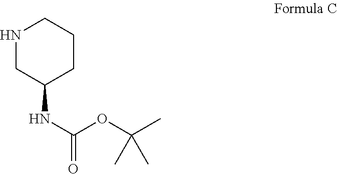
or its pharmaceutically acceptable salt using organic base in presence of suitable solvent to obtain substantially pure Boc-Linagliptin of Formula III 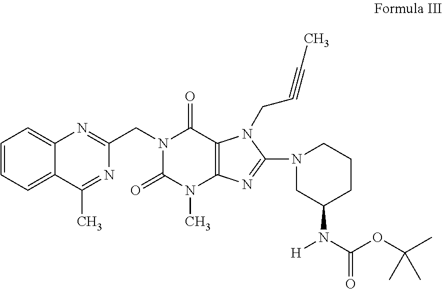 [SMILES: CC#CCn1c(N2CCC[C@@H](NC(=O)OC(C)(C)C)C2)nc2c1c(=O)n(Cc1nc(C)c3ccccc3n1)c(=O)n2C]
b) deprotection of Boc-linagliptin using trifluoroacetic acid in presence of dichloromethane to obtain crude linagliptin and (b) treatment with suitable acid base to obtain substantially pure Linagliptin.

33. The according to claim 32, wherein the organic base is selected from the group of triethyl amine (Et3N), trimethyl amine (Me3N), pyridine, tributylamine and diisopropyl ethyl amine (DIPEA).

34. The process according to claim 32, wherein suitable solvent is selected from dimethyl formamide, dimethylacetamide, dimethyl sulfoxide and water or mixtures thereof.

35. The process according to claim 32, wherein the substantially pure Boc-Linagliptin has the purity more than 95% by HPLC.

36. The process according to claim 32, wherein the 3-(R)-Bocaminopiperidine or its salt is 1 to 1.5 molar equivalents for the equivalent of 8-bromo xanthine of Formula II or its salt.

37. The process according to claim 32, wherein the base is 1 to 4 molar equivalents per the equivalent of the compound of Formula II or its salt.

38. The process according to claim 32, wherein the condensation reaction is performed at elevated temperature of about 45° C. to 11° C.

39. The process according to claim 25, wherein the Linagliptin is substantially free of “impurity-A”.
